(19) United States
(12) Patent Application Publication
(10) Pub. No.: US 2025/0277250 A1
(43) Pub. Date: Sep. 4, 2025

(54) REACTION MEDIUM AND ASSOCIATED METHOD FOR DETECTION OF A TARGET MICROORGANISM

(71) Applicant: BIOMERIEUX, Marcy L'Etoile (FR)

(72) Inventors: Thomas JUNILLON, Craponne (FR); Benoit MALLEN, Grezieu la Varenne (FR); Patrice CHABLAIN, Voiron (FR); David MOSTICONE, Sainte Concorce (FR)

(21) Appl. No.: 18/858,962

(22) PCT Filed: Apr. 25, 2023
(86) PCT No.: PCT/FR2023/000050

§ 371 Date: Oct. 22, 2024

(30) Foreign Application Priority Data
Apr. 26, 2022 (FR) FR2203888

(51) Int. Cl. C12Q 1/04; C12Q 1/10; G01N 33/543; G01N 33/569

(52) U.S. Cl. CPC C12Q 1/045; C12Q 1/10; G01N 33/54346; G01N 33/56916; G01N 2333/245

(57) ABSTRACT

The invention relates to a gelled reaction medium for the detection, identification, enumeration and/or isolation of at least one target microorganism in a simple that may contain same, comprising at least one binding partner specific to a component of a target microorganism or of a component derived from said microorganism, coupled to at least one nanoparticle to form at least one binding conjugate.

### TECHNICAL FIELD

The present invention relates to the field of microbiological control in a broad sense, such as the microbiological control of a sample of industrial or clinical origin. More particularly, the present invention relates to a reaction medium and to the associated method for detecting, identifying, counting and/or isolating a target microorganism.

### PRIOR ART

The microbiological control of samples of various origins requires the implementation of techniques which allow the detection-for example for identification and/or counting and/or biochemical characterization purposes-of microorganisms, and which must be rendered as quickly as possible in terms of results.

In the medical field, it is necessary to anticipate and diagnose the risk of infection: the faster and more accurate the diagnosis, the more effective the management of patients, and the lower the risk of transmission. The approach is similar for animal health in the veterinary field.

In the agrifood sector, the issues are identical. However, a distinction is made between:
pathogenic microorganisms such as Shiga-toxin-producing bacteria (STEC), Salmonella, Listeria, Cronobacter, Bacillus, Staphylococcus, whose detection applies to starting materials, intermediate products and finished products sold,
non-pathogenic microorganisms, used as quality indicators of the production process, from starting materials to finished products, throughout the chain,
bacteria of technological interest, such as ferments,
microorganisms which are markers of contamination.
   Rapid and accurate detection of suspected contamination (within food batches) enables them to be monitored, and thus enables corrective actions to be taken quickly.

Technically, one of the main difficulties is to be able to isolate the bacterium of interest in order to identify it. In general, microbiological analysis is a two-step process. The first step is a detection phase, which may involve a number of technologies such as culture media, immunoassays and molecular biology. This may be followed, notably in the agrifood sector, by a confirmation phase to confirm the presence of the pathogen of interest and comply to the standards in force in this field. The confirmation step thus involves additional steps, and here again, requires the isolation of the bacterium of interest.

Thus, in the case of Shiga-toxin-producing E. coli (STEC), diagnosis relies on the use of selective, chromogenic agars such as SMAC (Sorbitol MacConkey Agar), RMAC (Rhamnose MacConkey Agar) or Rainbow Agar O157, to select the growth of certain bacteria and stain the strains of interest. Ultimately, however, none of these agars is sufficiently specific for STEC without performing culturing to isolate and confirm the identity of the strain using PCR-type molecular biology tests. Specifically, their pathogenicity involves the expression of several virulence genes, notably stx1 and stx2 coding for the two types of Shiga-toxins: STX1 and STX2. These two toxins act by inhibiting protein synthesis in eukaryotic cells, ultimately leading to apoptosis. To identify pathogenic STEC accurately, it is essential to perform PCR for the genes stx1, stx2 or eae (another virulence factor). These processes are thus time-consuming and laborious, and the germ carrying both genes (stx and eae) is not systematically found in culture.

Immunological methods are also available. A large number of tests are available for the detection of E. coli O157:H7 in food and/or environmental samples. These systems comprise classic microplate ELISA tests, one-step immunological systems and fully automated systems.

“One-step” immunological methods are widely used in industry, due to their rapid implementation and their simplicity. Many of these systems are based on the principle of immuno-chromatography. The device consists of a plastic support containing a membrane impregnated with gold or latex particles coated with antibodies specific to E. coli O157:H7 (i.e. O157 and possibly H7), a sample well and a test and control window. The appearance of a colored line in the test window indicates a positive result, signaling the probable presence of E. coli O157 in the food. Mention may be made, for example, of the “VIP EHEC” test (BioControl, Montesson, France) which makes it possible, after enrichment for several hours, to visualize whether or not a given sample is contaminated with E. coli O157. However, this method does not make it possible to locate the E. coli O157 strain in the sample, nor its pathogenicity.

ELISA/ELFA systems are immunological methods which provide results in 2 hours in microplates, after an enrichment phase (usually lasting 24 hours). The company bioMérieux® has developed automated ELISA (ELFA) kits based on VIDAS® technology. In the event of a positive test, these methods have the drawback of not directly isolating the suspect colony. It is thus necessary to culture the sample in order to isolate the positive colony, and to confirm by PCR that this bacterium is indeed the one that caused the test to be positive.

ELISA methods are also available for the detection of non-O157 STECs, which are based essentially on the detection of Shiga-toxin production, possibly after an enrichment step. Some of these methods use monoclonal or polyclonal antibodies directed against the STX toxin, and visualization with alkaline phosphatase-linked antibodies. Among the ELISA kits available, mention may be made of “Premier EHEC” targeting Shiga-toxins (Meridian Diagnostics Inc., USA). Kits using the reverse passive agglutination assay (RPLA) technique suitable for STX toxin detection have also been developed and sold. This technique uses antibody-coated beads which interact with Shiga-toxins, producing a diffuse layer at the base of the wells in which the culture supernatant is present. These methods then require isolation of the positive colony, followed by a confirmation step.

The immunomagnetic separation (IMS) method has also been adapted for the detection and isolation of strains belonging to the five major STEC serogroups. It is performed in liquid medium, using magnetic beads coated with antibodies directed against antigens O26, O111, O103 and O145. However, strains isolated by this method must be confirmed for the presence of the stx gene before they can be considered STEC strains.

There is thus a real need to develop a reliable and rapid method for isolating and identifying target bacteria, notably pathogenic bacteria.

### SUMMARY OF THE INVENTION

A first subject of the invention relates to a gelled reaction medium for detecting, identifying, counting and/or isolating at least one target microorganism in a sample liable to contain same, comprising at least one specific binding partner of a component of a target microorganism or of a component derived from said microorganism, coupled to at least one nanoparticle to form at least one agglutinating conjugate.

The component used for detecting the target microorganism is a component released by said microorganism into the reaction medium. It may be a constituent of said target microorganism, such as a lipopolysaccharide (LPS), or a component produced by the target microorganism, such as a toxin.

Very advantageously, the medium thus makes it possible to localize, on the culture medium, the target colony releasing said component. This improves the detection of the target microorganism and saves very valuable time in the field of microbiological control, notably during the confirmation step.

Preferentially, the conjugate comprises a colloidal nanoparticle with optical properties.

Advantageously, the medium according to the invention comprises a toxin inducer. Thus, the medium according to the invention provides a highly advantageous means of detecting Shiga-toxin-producing E. coli (STEC) strains.

Another subject of the invention relates to a detection method for identifying, counting and/or isolating a target microorganism in a sample liable to contain same, comprising the following steps:
placing said sample in contact with a reaction medium according to the invention
incubating
detecting the presence of said target microorganism.

Advantageously, detection takes place via the appearance of a halo around the target microorganism on the reaction medium.

### DESCRIPTION OF THE FIGURES

FIG. 1 is a photograph of a culture medium according to the invention for detecting the strain E. coli O26, comprising a phage protein coupled to a gold nanoparticle.

FIG. 2 is a photograph of a culture medium according to the invention for detecting the strain E. coli O111, comprising an antibody coupled to a gold nanoparticle.

FIG. 3 is a photograph of a culture medium according to the invention for detecting an STX1 toxin-producing target bacterium, comprising an anti-STX1 antibody coupled to a gold nanoparticle, in the presence of ciprofloxacin as toxin inducer.

FIG. 4 is a photograph of a culture medium according to the invention for detecting an STX1 toxin-producing target bacterium, comprising an anti-STX1 antibody coupled to silver nanoparticles, in the presence of mitomycin as toxin inducer.

FIG. 5 is a photograph of a culture medium according to the invention for detecting an STX1 and/or STX2 toxin-producing target bacterium, comprising an anti-STX1 antibody coupled to a silver nanoparticle, an anti-STX2 antibody coupled to a gold nanoparticle, in the presence of mitomycin.

FIG. 6 is a photograph of a culture medium according to the invention for detecting an STX1 and/or STX2 toxin-producing target bacterium, comprising an anti-STX1 or anti-STX2 antibody coupled to the same gold nanoparticle, in the presence of ciprofloxacin as toxin inducer.

### DETAILED DESCRIPTION OF THE INVENTION

Certain terms and expressions used in the context of the invention are detailed hereinbelow.

A first subject of the invention relates to a gelled reaction medium for detecting, identifying, counting and/or isolating at least one target microorganism in a sample liable to contain same, comprising at least one specific binding partner of a component of a target microorganism or of a component derived from said microorganism, coupled to at least one nanoparticle to form at least one agglutinating conjugate.

In other words, the invention relates to a gelled reaction medium for detecting, identifying, counting and/or isolating at least one target microorganism in a sample liable to contain same, comprising at least one agglutinating conjugate formed by at least one specific binding partner of a component of a target microorganism or of a component derived from said microorganism, coupled to at least one nanoparticle. Very surprisingly, it was found that it was possible to detect a target microorganism using a gelled reaction medium comprising an agglutinating conjugate.

The term “reaction medium” refers to a medium comprising all the elements required for the expression of a metabolism, for survival and/or growth of microorganisms. This reaction medium may either be a microbiological culture medium, or a microbiological visualization medium. In the latter case, the microorganisms may first be cultured in another medium. The reaction medium may also be placed in contact with an agar culture medium. It may be placed under or on top of the culture medium which enables the growth of the target microorganisms. The reaction medium may be added after incubation of the culture medium. Prior to use, the reaction medium may be dehydrated. The reaction medium may be in pad form.

According to the present invention, the reaction medium is gelled. It is in solid or semi-solid form. Agar is the conventional gelling agent used in microbiology for the culture of microorganisms, but other gelling agents can also be used, for instance gelatin, agarose and also other natural or artificial gelling agents. Thus, contrary to all expectation, the conjugate is capable of forming a network with the target component in a gelled medium. Surprisingly also, this network can be visually detected by the formation of a halo, without the need to reduce the hardness of a conventional semi-solid reaction medium. Thus it is not necessary to modify the physicochemical properties of the reaction medium, such as the hardness, to allow diffusion of the target components on the one hand, and reaction with the conjugates on the other.

A number of preparations are commercially available, for instance Columbia agar, Trypticase soy agar, MacConkey agar, Mueller Hinton agar or more generally those described in the Handbook of Microbiological Media. These media may serve as a basis for the reaction medium according to the invention. The reaction medium may also comprise optional additives such as, for example, amino acids, peptones, one or more growth factors, carbohydrates, nucleotides, minerals, vitamins, one or more selective agents, inducers, toxin inducers, buffers, etc. The term “selective agent” refers to any compound that is capable of preventing or slowing down the growth of a “non-target” microorganism, i.e. one other than the target microorganism(s). The term “inducer” refers to a compound that is capable of inducing the expression of a compound, such as an enzyme or toxin, which would normally remain unexpressed.

Said reaction medium may also comprise a colorant. As a guide, Evans blue, neutral red, sheep's blood, horse's blood, an opacifier such as titanium oxide, nitroaniline, malachite green, and brilliant green may be mentioned as colorant. When the reaction medium according to the invention also comprises an enzyme substrate specific for an enzymatic activity of at least one target microorganism, a chromogenic and/or fluorogenic substrate is preferably used. The term “chromogenic and/or fluorogenic substrate” refers to a substrate enabling the detection of an enzymatic or metabolic activity of the target microorganisms/microorganisms of interest by means of a detectable signal. The reaction medium according to the invention may also include a pH indicator, sensitive to the pH variation induced by the consumption of the substrate and revealing the metabolism of the target microorganisms. Said pH indicator may be a chromophore or a fluorophore. Examples of chromophores that may be mentioned include bromocresol purple, bromothymol blue, neutral red, aniline blue and bromocresol blue.

A person skilled in the art may also use a Petri dish divided into segments, such as a bi-dish or tri-dish, enabling easy comparison of several media, comprising different substrates or different selective mixtures, on which the same biological sample has been deposited.

According to the present invention, the reaction medium comprises a specific binding partner of a component of a target microorganism or of a component derived from said microorganism, coupled to a nanoparticle. The specific binding partner is chosen from antibodies, all types of Fab fragments, recombinant proteins, phages, phage proteins, oligonucleotides, aptamers, Affimers or any other ligand or anti-ligand that is well known to a person skilled in the art. According to the present invention, the reaction medium comprises a specific binding partner which is not bound to said component of the target microorganism or to the component derived from said microorganism. It is only on using said medium, i.e. on inoculating the sample onto the reaction medium, that the specific binding partner can bind to the component of a target microorganism or a component derived from said microorganism.

Preferentially, the antibody is a monoclonal antibody or a monoclonal antibody fragment.

According to the present invention, the binding partner is specific to a component of a target microorganism. The component of the target microorganism is a component released by said microorganism. The component may thus be an element originating from the surface of the bacterium, such as a protein, a lipopolysaccharide (LPS) or a flagellum. It may also be an element internal to the bacterium, such as RNA or an intracellular protein, which may be detected when part of the bacterial colony dies during its growth. In a particular embodiment, the binding partner is specific to the RNA of the target microorganism. In this embodiment, the binding partner is composed of at least two different primers hybridizing complementarily to the target RNA.

In another particular embodiment, the binding partner is specific to a component derived from said microorganism. This component may be a molecule of interest produced by the target microorganism, such as a protein, an antibiotic, an antimicrobial resistance molecule, protease-type enzymes, lipases or glucosidases.

According to the present invention, the binding partner is coupled to a nanoparticle. This final complex is called a conjugate. In the same reaction medium, it is possible to have conjugates having binding partners of a different nature, coupled to nanoparticles which are themselves of a different nature.

The term “nanoparticle” refers to particles of nanometric size. Nanoparticles may be chosen from gold, iron, silver, copper, carbon, latex, silicon and aluminum. Preferentially, the nanoparticles are colloidal nanoparticles chosen for their optical properties, i.e. their ability to be distinguished when a network is formed. Even more preferentially, the nanoparticle is chosen from gold, silver and copper. Thus, when the nanoparticles are gold, they change color, for example from red to gray, when they form a network. When they are not aggregated, the wavelength of the light absorbed is in the red region around 530 nm. When they are aggregated, the absorbed wavelength changes from red to blue/gray around 600 to 700 nm. In a particular embodiment, several nanoparticles of different colors may be used together. The networks thus formed enable several components of the target microorganism to be distinguished.

Preferentially, the nanoparticles have a size of between 10 and 200 nm. Preferentially, the nanoparticles have a size of between 20 and 90 nm, allowing better mobility of the conjugates in the reaction medium.

Advantageously, the nanoparticles make it possible to reduce the amount of binding partners required for agglutination formation. Thus, the concentration of binding partners required to produce a reaction medium according to the invention requires 100 to 1000 times fewer binding partners than a nanoparticle-free medium.

Preferentially, the required amount of binding partners corresponds to the amount required to cover at least half the nanoparticle surface, and even more preferentially to cover between a third and half the nanoparticle surface. This proportion enables the agglutinating conjugate to form a network in the gelled reaction medium.

Advantageously, the nanoparticles may allow visualization of agglutination around a bacterial colony whose size is not yet visible to the naked eye. Detection may thus be earlier. Advantageously, the nanoparticles may allow visualization of agglutination around a bacterial colony whose translucent appearance does not allow detection by an automatic reader. Detection may thus be facilitated.

Coupling of the nanoparticle to the binding partner may take place by either direct attachment or indirect attachment. Direct attachment means attachment by either adsorption or by covalent bonding. Indirect attachment means attachment by the interaction of ligands/anti-ligands, for instance biotin/streptavidin or other pairs that are well known to a person skilled in the art. Depending on the type of binding chosen, a person skilled in the art will adapt the physicochemical conditions of the reaction medium, notably its pH.

According to the present invention, the conjugate is agglutinating, i.e. it causes the formation of an agglutination network in the presence of a component of a target microorganism or of a component derived from said microorganism. As the component is multi-epitope, several conjugates will bind to this component and form agglutination. The term agglutination refers to the result of an interaction between at least one component of a target microorganism or at least one component derived from said microorganism with binding partners coupled to a nanoparticle. Agglutination reactions comprise immunological reactions, such as antigen-antibody reactions, or more generally, specific interactions between two molecules. Through this interaction, components and conjugates aggregate, adhere to each other and form a network in the reaction medium. In practice, several parameters influence the ability of a conjugate to be agglutinating in a gelled medium, such as mainly:
the porosity of the gelled medium
the size of the nanoparticles
the amount of binding partners
the amount of nanoparticles.
   These parameters should thus be adapted to ensure satisfactory agglutination for detection. Advantageously, the network formed by said specific reaction is then detected either visually or automatically using an optical system. The colony of the target microorganism is thus located. Preferably, the network forms a halo in the gelled reaction medium that can be detected either visually or using an optical system. The network or halo thus circumscribes the colony, which may then be advantageously differentiated and/or identified within a population.

For the purposes of the present invention, the term “at least one target microorganism” refers to at least one microorganism that is to be detected and/or identified and/or counted. Preferentially, the microorganism is chosen from Escherichia coli, Shiga-toxin-producing Escherichia coli, Shigella, Salmonella typhimurium, Salmonella enteritidis, Pseudomonas, Bacillus cereus group, Enterococcus faecalis, Enterococcus faecium, Staphylococcus epidermidis, Staphylococcus aureus MSSA, Staphylococcus aureus MRSA, and Streptococcus agalactiae. Preferably, the microorganism is chosen from E. coli strains, and preferentially from Shiga-toxin-producing E. coli (STEC) strains. Enterohemorrhagic E. coli (EHEC) are strains representing a subgroup of Shiga toxin STX-producing Escherichia coli (STEC), which have acquired the eae gene and cause hemolytic uremic syndrome. The simultaneous possession of these two virulence factors makes this pathovar highly virulent for humans. These notably include serogroups O26, O45, O80, O103, O111, O121, O145 and O157.

In a particular embodiment, the reaction medium comprises an inducer that promotes or enhances the expression of a molecule of interest produced by the target microorganism. This molecule of interest may be a protein, an antibiotic, an antimicrobial resistance molecule, a protease-type enzyme, a lipase or a glucidase. Thus, the present invention enables the characterization or selection of microbial strains relative to their ability to produce these molecules of interest. In a particular embodiment, the present invention may relate to the field of bioproduction, and more particularly the production of recombinant proteins from a genetically modified microorganism, for which it is required to identify the producing clones. The present invention can thus clearly show that the recombinant protein is being produced. In the presence of a halo, it is also possible to estimate the amount produced. In another particular embodiment, the present invention enables the detection of resistant pathogenic bacteria.

In a particular embodiment, the reaction medium comprises a toxin inducer. The toxin inducer causes stress in the bacterium, triggering the lytic cycle of prophages in the bacterium and thus stimulating the production of toxins, which are released. By way of illustration, mention may be made of the toxins secreted by Staphylococcus aureus or the Shiga-toxins secreted by STEC strains. There are two main types of Shiga-toxins: STX1 and STX2, which themselves have numerous variants. To date, STX1 has 4 subtypes STX1a, STX1c, STX1d, STX1e, and STX2 has 12: STX2a, STX2b, STX2c, STX2d, STX2e, STX2f, STX2g, STX2h, STX2i, STX2j, STX2K, STX2l. Advantageously, the toxin inducer is chosen from antibiotics or physicochemical stress. Mention may be made, as antibiotics, of trimethoprim, sulfamethoxazole, norfloxacin, azithromycin, gentamicin, polymyxin B, chloramphenicol, streptomycin, chlortetracycline, oxytetracycline, tylosin, mitomycin C, carbodox, oliquindox, rifampicin, imipenem, ciprofloxacin, cotrimoxazole, penicillin G, and lincomycin. In a preferred embodiment, the medium according to the invention comprises ciprofloxacin in a concentration of between 0.005 and 0.030 mg/l. In another preferred embodiment, the medium according to the invention comprises mitomycin C in a concentration of between 0.10 and 0.5 mg/l.

The toxin inducer may also be a physicochemical stress produced, for example, by the addition of salt or EDTA, or by a pH change or UV stress. The stress may also be induced, for example, by the addition of noradrenaline.

Said target microorganism is liable to be present in a sample. The term “sample” refers to a small part or small amount isolated from an entity for analysis. The sample may be of industrial origin, such as, without this list being limiting, an air sample, a water sample, a sample collected from a surface, a manufactured part or product, or a food product. Food samples that may be mentioned include, without this list being limiting, samples of dairy products (yoghurts, cheeses, etc.), meat, fish, eggs, fruit, vegetables, water and beverages (milk, fruit juice, carbonated soft drink, etc.). Finally, a food sample may be derived from animal feed, notably such as animal or vegetable meal. The sample may be of biological origin, either animal or human. It may then correspond to a sample of biological fluid (stools, urine, whole blood, serum, plasma, cerebrospinal fluid, organic secretion, etc.), an external sample (skin, nose, throat, etc.) or a tissue sample or isolated cells. The sample may be used as is or, prior to analysis, may undergo preparation such as enrichment, extraction, concentration or purification, using methods known to a person skilled in the art. The reaction medium according to the invention may be all the more interesting when the sample is polymicrobial or loaded with ancillary flora, for instance food samples such as raw milk cheese, or stools in clinical samples.

Another subject of the present invention relates to a diagnostic kit for the preparation of a reaction medium according to the invention, comprising
an agglutinating conjugate as described in the present invention
a gelling medium.

Advantageously, the reaction medium may be manufactured extemporaneously using a kit according to the invention. This enables the use of the reaction medium as a unit, and increases its stability.

Thus, another subject of the present invention relates to the process for obtaining a reaction medium according to the invention, comprising the steps of placing an agglutinating conjugate in contact with a gelling medium to form the reaction medium according to the invention. The conjugate is prepared by coupling the binding partner to the nanoparticle. These coupling methods are well known to a person skilled in the art (Nicholas G. Welch et al., 2017). The conjugate is then added to the gelled medium which has been supercooled. The whole is then homogenized and poured into the Petri dish.

Another subject of the present invention relates to an in vitro microbiological culture method, in which microorganisms liable to be present in a sample are inoculated in or on a culture medium according to the invention. The inoculated culture medium is incubated under suitable conditions known to those skilled in the art. Inoculation is performed using conventional microbiology techniques. It may also be performed in the mass, i.e. by inclusion.

Another subject of the present invention relates to a method for detecting, identifying, counting and/or isolating at least one target microorganism in a sample liable to contain same, comprising the following steps:
bringing said sample into contact with a reaction medium according to the invention
incubating
detecting the presence of said target microorganism.

The term “detection” refers to detection with the naked eye or with the aid of an optical apparatus of the existence of growth of target microorganisms, preferably target bacteria. When the reaction medium from which the target microorganisms are to be detected comprises a chromogenic or fluorogenic substrate, detection may be performed with the aid of an optical apparatus for fluorogenic substrates, or with the naked eye or with the aid of an optical apparatus for chromogenic substrates.

The term “identification” refers to the determination of the genus and/or species and/or group to which a target microorganism belongs.

The term “counting at least one target microorganism” refers to the counting/quantification of the number of target microorganisms, for example the number of bacterial colonies when the target microorganism is a bacterium.

The term “isolation” refers to the production of different colonies spaced apart from each other.

Microbiological testing corresponds to the analysis of a sample for the purpose of detecting microorganisms that are liable to be present within said sample. Thus, very surprisingly, it was found that a medium according to the invention made it possible to detect a target bacterium and to isolate it. Advantageously, the medium according to the invention may thus make it possible to locate and select a target microorganism from a mixed population present on a non-selective or insufficiently selective reaction medium. Preferentially, the method according to the invention enables detection by the appearance of a halo around said target microorganism on the reaction medium according to the invention. In other words, detection is performed by observing the appearance of a halo around the target microorganism on the reaction medium.

Without this being limiting, it appears that the medium according to the invention is particularly suitable for the detection of E. coli. Thus, according to a particular embodiment, the present invention relates to a gelled reaction medium for detecting, identifying, counting and/or isolating an E. coli strain, comprising a phage protein specific for the LPS of said strain coupled to a nanoparticle to form a conjugate.

In this particular embodiment, the amount of LPS-specific phage protein makes it possible to cover at least a third of the nanoparticle surface.

Preferentially, in this particular embodiment, the nanoparticle is gold, with a size of between 20 and 90 nm and a concentration of between 10¹⁰ and 10¹² nanoparticles/ml of reaction medium.

In this embodiment, the reaction medium is formed from a known culture medium for the culture of E. coli strains, such as the Applicant's TBX or chromID® Coli media, to which the agglutinating conjugates have been added.

In another particular embodiment, the present invention relates to a gelled reaction medium for detecting, identifying, counting and/or isolating an E. coli strain, comprising a monoclonal antibody specific for said strain coupled to a nanoparticle to form a conjugate.

Preferentially, in this particular embodiment, the amount of monoclonal antibody makes it possible to cover at least half the nanoparticle surface, and even more preferentially to cover between a third and a half of the nanoparticle surface.

Preferentially, in this particular embodiment, the nanoparticle is gold, with a size of between 20 and 90 nm and a concentration of between 10¹⁰ and 10¹² nanoparticles/ml of reaction medium.

In another particular embodiment, the present invention relates to a gelled reaction medium for detecting, identifying, counting and/or isolating a Shiga-toxin-producing E. coli strain comprising at least one toxin inducer, at least one antibody specific for Stx1 and/or Stx2, said at least one antibody being coupled to a nanoparticle to form an agglutinating conjugate. In other words, the present invention relates to a gelled reaction medium for detecting, identifying, counting and/or isolating a Shiga-toxin-producing E. coli strain comprising at least one toxin inducer, at least one agglutinating conjugate formed by at least one antibody specific for Stx1 and/or Stx2 coupled to a nanoparticle.

Advantageously, this embodiment makes it possible to locate a Shiga-toxin-producing E. coli strain by the formation of a halo around said strain. Specifically, the conjugates formed from antibodies coupled to the nanoparticles have agglutinated around said colony. It is thus easy to visualize the target colony.

In this embodiment, said at least one antibody has an that amount makes it possible to cover at least half of the nanoparticle surface and preferentially at least a third of the nanoparticle surface. In these particular embodiments, the medium comprises ciprofloxacin, in a concentration of between 0.005 and 0.030 mg/l. In a variant of these particular embodiments, the medium comprises mitomycin C, in a concentration of between 0.10 mg/l and 0.50 mg/l.

Preferentially, in these variants, the nanoparticle is a gold nanoparticle between 20 and 90 nm in size and in a concentration of between 10¹⁰ and 10¹² nanoparticles/ml of reaction medium.

When the medium according to the invention comprises binding partners of several toxins, the present invention is particularly advantageous for discriminating in the same sample between bacteria producing Shiga-toxin STX1 or STX2 and bacteria producing both types of Shiga-toxins STX1 and STX2.

The present invention is particularly advantageous for facilitating the detection of STECs in a polymicrobial sample. Indeed, without the present invention, which enables the colony of interest to be located, the reference method ISO 16136 specifies that it is necessary to test up to 50 colonies by a molecular method to confirm the presence of a STEC. In view of the large number of colonies on the dish and the poor representation of the target microorganism, the person picking the colonies for confirmation purposes might never pick the target microorganism. Thus, the present invention saves time in carrying out microbiological testing. It is particularly advantageous for samples loaded with ancillary flora, where the large number of colonies on Petri dishes may lead to a risk of false negatives.

### EXAMPLES

### Example 1: Detection of E. coli O26 With a Medium According to the Invention Comprising a Phage Protein Coupled to a Gold Nanoparticle

### Preparation of the Gold Nanoparticles

The 40 nm nanoparticles are manufactured by reducing gold chloride with sodium citrate (method described by Turkevich and Frens in 1951). Thus, 20 nm gold nanoparticles are manufactured using a solution of gold chloride diluted in distilled water, to which trisodium citrate has been added, and then boiled. The presence of an absorbance peak at 517-519 nm makes it possible to confirm the correct particle size. From these 20 nm particles, 40 nm particles are synthesized. To do this, the 20 nm particles are diluted in distilled water and then boiled with the addition of trisodium citrate and gold chloride. The absorbance peak is then observed at 524-526 nm in the cold state, reflecting the increase in the size of the particles.

### Preparation of the Conjugate

The phage evaluated in this assay is Phage Eco O26 BP1, directed against the LPS of Escherichia coli O26. This phage is from the Applicant's collection. Adsorption of the phage protein onto the nanoparticles is performed taking into account the value of its isoelectric point and according to methods known to a person skilled in the art (Nicholas G. Welch et al.): The above phage is coupled as follows
dilution of 270 μg of phage protein in 3 ml Tris HCl (0.025 M) pH 6
addition of 30 ml of gold at an optical density of 1, i.e. 7.7×10¹⁰ nanoparticles/ml, previously adjusted to pH 6 with 0.1 M K₂CO₃ solution
stirring for 20 minutes
passivation by adding 3 ml of 10% BSA
centrifugation at 8600 g for 30 min
removal of the supernatant and assay of the resulting concentrate using a spectrophotometer.

### Preparation of the Reaction Medium According to the Invention

The conjugate produced is added to supercooled agar (chrom ID coli-ref 42017 bioMérieux) at 50° C. so as to obtain a concentration of nanoparticles with an optical density of 3 using a spectrophotometer. 18 ml of medium per dish are then poured. The dishes are then dried.

### Inoculation

In this example, a strain of E. coli O26 and a strain of E. coli O111 (Applicant's collection) are inoculated and incubated for 72 h at 37° C.

### Results

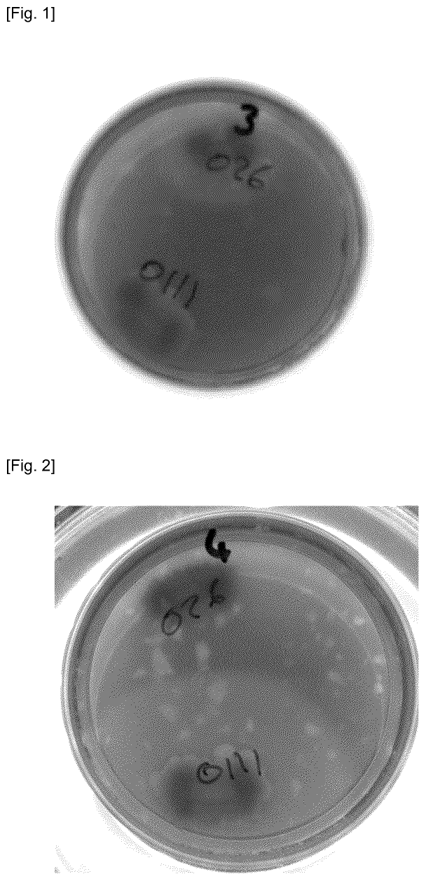

A gray halo is visually observed around the colony with LPS O26 (FIG. 1). This turn to gray is due to a network formed between the LPS released by the colonies and the conjugates present in the agar.

### Example 2: Detection of E. coli O111 With a Medium According to the Invention Comprising an Antibody Coupled to a Gold Nanoparticle

### Preparation of the Gold Nanoparticles

The 40 nm gold particles are manufactured as shown in Example 1.

### Preparation of the Conjugate

The 2H5E9 IgG anti-E. coli O111 monoclonal antibody is from the Applicant's collection.

The antibody is adsorbed onto the nanoparticles as follows:
dilution of 90 μg of antibody in 3 ml Tris HCl (0.025 M) pH 6.5
addition of 30 ml of gold at OD 1 previously adjusted to pH 8 with 0.1 M K₂CO₃ solution
stirring for 20 minutes
passivation by adding 3 ml of 10% BSA
centrifugation at 8600 g for 30 min
removal of the supernatant and assay of the resulting concentrate using a spectrophotometer.

### Preparation of the Reaction Medium According to the Invention

The conjugate produced is added to supercooled agar (chrom ID coli-ref 42017 bioMérieux) at 50° C. so as to obtain a concentration of nanoparticles with an optical density of 3 using a spectrophotometer. 18 ml of medium per dish are then poured. The dishes are then dried.

### Inoculation

In this example, a strain of E. coli O26 and a strain of E. coli O111 from the Applicant's collection are inoculated and incubated for 72 hours at 37° C.

### Result

There is no gray halo around colony O26, whereas it is clearly visible around colony O111 (FIG. 2), which makes it possible for the latter to be identified.

### Example 3: Detection of STX1 Toxin-Producing E. coli With a Medium According to the Invention Comprising an Anti-Stx1 Antibody Coupled to a Gold Nanoparticle and Ciprofloxacin as Toxin Inducer

### Preparation of the Gold Nanoparticles

The 40 nm gold particles are manufactured as described in Example 1.

### Preparation of the Conjugate

The antibody evaluated in this assay is the IgG antibody (ref ATCC 13C4 hybridoma CRL-1794) directed against the STX1 toxin.

The antibody is adsorbed onto the nanoparticles as follows:
dilution of 90 μg of antibody in 3 ml Tris HCl (0.025 M) pH 8
addition of 30 ml of gold at OD 1 previously adjusted to pH 8 with 0.1 M K₂CO₃ solution
stirring for 20 minutes
passivation by adding 3 ml of 10% BSA
centrifugation at 8600 g for 30 min
removal of the supernatant and assay of the resulting concentrate using a spectrophotometer.

### Preparation of the Medium According to the Invention

Agar supplementation:
The conjugate produced is added to supercooled agar (chrom ID coli ref 42017 bioMérieux) at 50° C. containing the toxin inducer, in this case ciprofloxacin at 10 ng/ml, so as to obtain a concentration of nanoparticles with an OD of 3. 18 ml of medium per dish are then poured. The dishes are then dried.

### Inoculation

Four strains from the Applicant's in-house collection, three of which possess the stx1 gene, are inoculated and incubated for 24 hours at 37° C.

### Conclusion

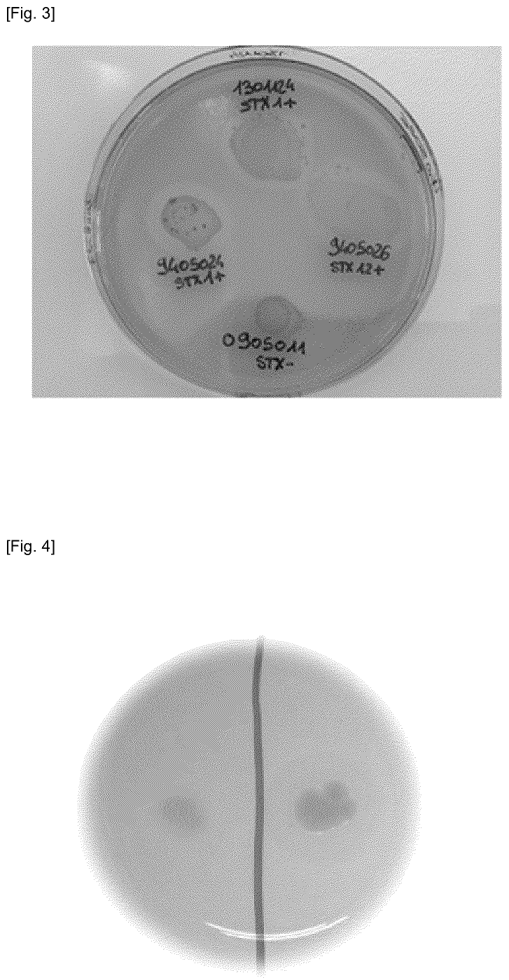

A gray halo is observed around the STX1 toxin-producing colonies (FIG. 3). The STX1 toxin is detected. This turn to gray is due to a network formed between the toxins released by the colonies and the conjugates present in the agar.

### Example 4: Detection of STX1 Toxin-Producing E. coli in a Medium According to the Invention Comprising an Anti-Stx1 Antibody Coupled to Silver Nanoparticles and Mitomycin as Toxin Inducer

### Preparation of the Silver Nanoparticles

The 40 nm silver nanoparticles were purchased from a supplier (Alfa Aesar; ref: J67090.AE).

### Preparation of the Conjugate

The antibody (ref ATCC 13C4 hybridoma CRL-1794) is adsorbed onto nanoparticles as described in Example 3.

### Preparation of the Medium According to the Invention

The conjugate produced is added to TBX supercooled agar (ref AEB622817 bioMérieux) at 50° C., which also contains a toxin inducer, in this case mitomycin C at 250 ng/mL, so as to obtain a concentration of nanoparticles with an OD of 3. The medium is then poured into the dishes and dried.

### Inoculation

Two strains from the Applicant's in-house collection, one of which possesses the stx1 gene, are inoculated and incubated for 24 hours at 37° C.

### Results

A gray halo is observed around the STX1 toxin-producing strain (FIG. 4, colony on the right). This turn to gray is due to a network formed between the toxin released by the colonies and the conjugates present in the agar.

### Example 5: Detection of Stx1 and/or Stx2 Toxin-Producing E. coli in a Medium According to the Invention Comprising an Anti-Stx1 Antibody Coupled to a Silver Nanoparticle, an Anti-Stx2 Antibody Coupled to a Gold Nanoparticle, in the Presence of Mitomycin C

### Preparation of the Silver or Gold Nanoparticles

The 40 nm silver nanoparticles were purchased from a supplier (Alfa Aesar; ref: J67090.AE).

The nanoparticles are manufactured according to Example 1.

### Preparation of the Conjugate

The 40 nm gold nanoparticles are coupled to the anti-stx2 antibody 9E4H11, from the Applicant's collection, according to the method described in Example 1 with a pH of 9.

The adsorption of the anti-stx1 antibody 13C4 onto the silver nanoparticles is performed according to Example 4.

### Preparation of the Medium According to the Invention

The conjugates are added to supercooled TBX agar (ref AEB622817 bioMérieux) at 50° C. containing the toxin inducer, in this case mitomycin C at 250 ng/ml, so as to obtain a concentration for each type of nanoparticle with an OD of 3. The medium is then poured into the dishes and dried.

### Inoculation

Two strains from the Applicant's collection, one with the stx1 gene and the other with the stx2 gene, are inoculated and incubated for 24 hours at 37° C.

### Results

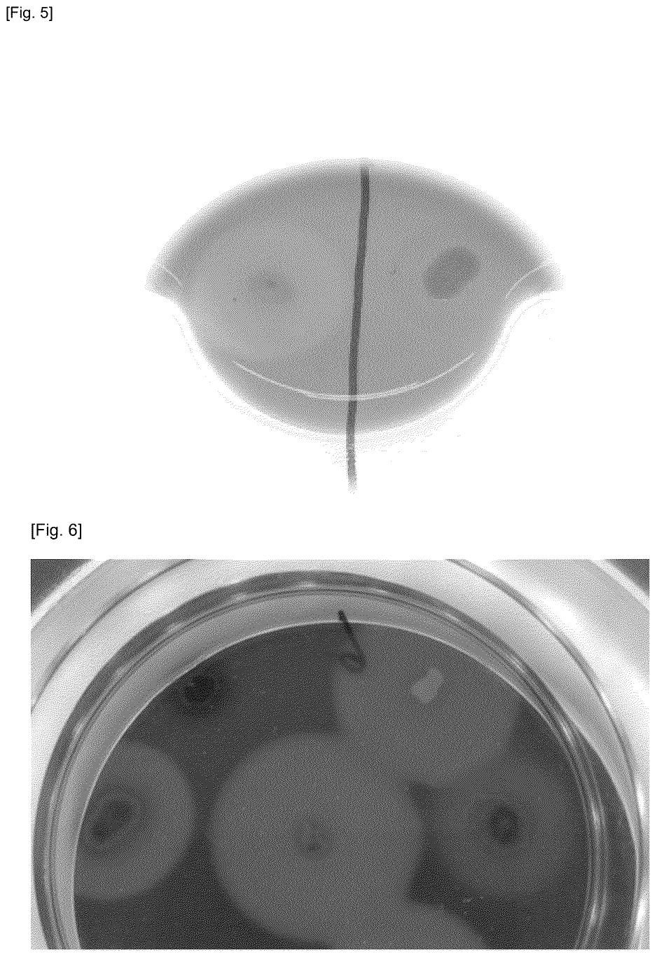

The reaction medium with the conjugates has a light red color. A halo can be observed around each of the two strains (FIG. 5). What is noticeable is that the color of the halo varies according to the toxin produced. Specifically, in the case of STX2 toxin production, a network is formed between the toxin and the gold nanoparticles, causing the gold nanoparticles to turn from red to gray, resulting in a yellow halo due to the presence of the silver nanoparticles that have not aggregated (colony on the left in FIG. 5). In the case of STX1 toxin production, a network is formed between the toxin and the silver nanoparticles, causing the silver nanoparticles to turn from yellow to gray and producing a more intense red halo (light red+gray) due to the presence of the gold nanoparticles that have not aggregated (colony on the right in FIG. 5).

### Example 6: Detection of an Stx1 and/or Stx2 Toxin-Producing Target E. coli in a Medium According to the Invention Comprising an Anti-Stx1 or Anti-Stx 2 Antibody Coupled to the Same Gold Nanoparticle in the Presence of Ciprofloxacin as Toxin Inducer

### Preparation of the Nanoparticles

The 40 nm nanoparticles are manufactured according to Example 1.

### Preparation of the Conjugates

The antibodies evaluated in this assay are the 13C4 antibody directed against the STX1 toxin and the 9E4H11 antibody directed against the STX2 toxin.

This mixture is adsorbed onto the nanoparticles according to the preceding examples at a pH of 8.

### Preparation of the Medium According to the Invention

The conjugate produced is added to supercooled TBX agar (ref AEB622817 bioMérieux) at 50° C. containing the toxin inducer, in this case ciprofloxacin at 10 ng/ml, so as to obtain a nanoparticle concentration with an OD of 3. 18 ml of medium per dish are then poured. The dishes are then dried.

### Inoculation

Five strains with or without stx1 and/or stx2 genes are inoculated and incubated for 24 hours at 37° C.

Conclusion: A gray halo is observed around the colonies producing STX1 and/or STX2 toxins (FIG. 6). This turn to gray is due to a network formed between the toxins released by the colonies and the conjugates present in the agar.

### Example 6: Detection of an STX1 Toxin-Producing E. coli Strain by Mass Inoculation

### Preparation of the Nanoparticles

The 40 nm nanoparticles are manufactured according to Example 1.

### Preparation of the Conjugates

The antibody used in this study is 13C4 directed against the STX1 toxin of Escherichia coli. Adsorption is performed as described in the preceding Examples.

### Preparation of the Medium According to the Invention

Add the conjugates produced in supercooled agar (TBX) at 50° C. containing the toxin inducer, in this case mitomycin C at 250 ng/ml, so as to obtain a concentration of nanoparticles with an OD of 3.

### Inoculation

In this Example, two strains, one producing STX1 toxin (O24), and the other not (O25), are inoculated in the mass and incubated for 24 hours at 37° C.

### Conclusion

In the case of the O24 colony, a gray halo is clearly present. This color shift is due to a network formed between the STX1 toxins released by the colonies and the conjugates present in the agar.

### BIBLIOGRAPHICAL REFERENCES

Nicholas G. Welch et al., “Orientation and characterization of immobilized antibodies for improved immunoassays (Review)”; Biointerphases 12, 02D301 (2017); https://doi.org/10.1116/1.4978435;

Turkevich et al., “A study of the nucleation and growth processes in the synthesis of colloidal gold”, 1951

1-27. (canceled)

28. A gelled reaction medium for detecting, identifying, counting and/or isolating at least one target microorganism in a sample liable to contain same, comprising at least one specific binding partner of a component of a target microorganism or of a component derived from said microorganism, coupled to at least one nanoparticle to form at least one agglutinating conjugate.

29. The gelled reaction medium of claim 28, wherein the gelled reaction is a microbiological culture medium.

30. The gelled reaction medium of claim 28, wherein the gelled reaction is a microbiological visualization medium.

31. The gelled reaction medium as claimed in claim 28, wherein the binding partner is chosen from antibodies, aptamers, phage proteins and primers.

32. The gelled medium as claimed in claim 28, wherein the nanoparticle is a colloidal nanoparticle with optical properties.

33. The gelled medium of claim 28, wherein the nanoparticle is chosen from gold, silver and copper.

34. The gelled medium of claim 28, wherein the nanoparticles have a size of between 10 and 200 nm.

35. The gelled medium of claim 28, wherein the reaction medium is in contact with an agar culture medium.

36. The gelled medium of claim 28, wherein the medium comprises an inducer of the component derived from said microorganism.

37. The gelled medium of claim 36, wherein the medium comprises a toxin inducer.

38. The gelled medium of claim 37, wherein the toxin inducer is an antibiotic.

39. The gelled medium of claim 38, wherein the toxin inducer is ciprofloxacin in a concentration of between 0.005 and 0.030 mg/l.

40. The gelled medium of claim 11, wherein the toxin inducer is mitomycin C in a concentration of between 0.10 mg/l and 0.50 mg/l.

41. The gelled reaction medium of claim 1, for detecting, identifying, counting and/or isolating at least one target microorganism chosen from Escherichia coli, Shiga-toxin-producing Escherichia coli, Shigella, Salmonella typhimurium, Salmonella enteritidis, Pseudomonas, Bacillus cereus group, Enterococcus faecalis, Enterococcus faecium, Staphylococcus epidermidis, Staphylococcus aureus MSSA, Staphylococcus aureus MRSA, Streptococcus agalactiae.

42. The gelled reaction medium of claim 28, for detecting, identifying, counting and/or isolating an E. coli strain, comprising a phage protein specific for the LPS of said strain coupled to a nanoparticle to form an agglutinating conjugate.

43. The gelled reaction medium of claim 42, wherein the amount of LPS-specific phage protein makes it possible to cover at least half the surface of the nanoparticle.

44. The gelled reaction medium of claim 42, wherein the nanoparticle is made of gold, is between 20 and 90 nm in size and has a concentration of between 1010 and 1012 nanoparticles/ml of reaction medium.

45. The gelled reaction medium of claim 28, for detecting, identifying, counting and/or isolating an E. coli strain, comprising a monoclonal antibody specific for said strain coupled to a nanoparticle to form an agglutinating conjugate.

46. The gelled reaction medium as claimed in claim 21, for detecting, identifying, counting and/or isolating at least one Shiga-toxin-producing E. coli strain, comprising at least one toxin inducer, at least one antibody specific for STX1 and/or at least one antibody specific for STX2, said at least one antibody being coupled to a nanoparticle to form an agglutinating conjugate.

47. The gelled reaction medium as claimed in claim 22, for detecting, identifying, counting and/or isolating at least one Shiga-toxin-producing E. coli, comprising ciprofloxacin in a concentration of between 0.005 and 0.030 mg/l.
